(19) United States
(12) Patent Application Publication
(10) Pub. No.: US 2026/0165830 A1
(43) Pub. Date: Jun. 18, 2026

(54) PHAKIC REFRACTIVE LENSES

(71) Applicant: Medennium, Inc., Irvine, CA (US)

(72) Inventors: Seyul Ahn, Irvine, CA (US); Meilu Huang, Irvine, CA (US); Dmitrii Dementiev, Moscow (RU); Jian Liu, Dallas, TX (US); Xin Wei, Irvine, CA (US)

(21) Appl. No.: 19/417,154

(22) Filed: Dec. 11, 2025

Related U.S. Application Data
(60) Provisional application No. 63/734,011, filed on Dec. 13, 2024.
(60) Provisional application No. 63/734,433, filed on Dec. 16, 2024.

(51) Int. Cl. A61F 2/16

(52) U.S. Cl. CPC A61F 2/1605; A61F 2/1601

(57) ABSTRACT

The present disclosure pertains to the phakic refractive lenses such as toric lenses, an advanced posterior chamber floating phakic refractive lens (PRL) designed for the correction of severe refractive errors, including myopia, hyperopia, and astigmatism. This innovative lens can reduce or minimize complications commonly associated with traditional phakic intraocular lenses, such as intraocular pressure elevation, cataract formation, and iris pigment dispersion. The phakic refractive lenses such as the toric lenses described herein feature a buoyant, flexible structure with a central aperture to facilitate aqueous humor flow, creating a natural centering force that stabilizes the lens within the pupillary space.

### CROSS-REFERENCE TO RELATED APPLICATIONS

This application claims the benefit of priority to U.S. Provisional Application No. 63/734,011, titled “PHAKIC REFRACTIVE LENSES,” filed Dec. 13, 2024 as well as to U.S. Provisional Application No. 63/734,433, titled “PHAKIC REFRACTIVE LENSES,” filed Dec. 16, 2024. The entirety of each application referenced in this paragraph is incorporated herein by reference.

### BACKGROUND

### Field

This application generally relates to apparatus and methods for phakic refractive lenses such as, for example, floating phakic refractive lenses such as floating posterior chamber phakic refractive lenses designed to address the challenges of maintaining stability and alignment within the dynamic environment of the eye.

### Description of the Related Art

A phakic lens can be implanted in two primary locations: the anterior chamber—the space behind the cornea and in front of the iris and the posterior chamber—the space behind the iris and in front of the natural crystalline lens. A posterior chamber phakic refractive lens (PRL) can be surgically implanted in the posterior chamber for correcting ametropia or refractive errors, such as myopia and hyperopia. The implantation of a phakic refractive lens is a reversible surgical procedure available for the correction of severe refractive errors in myopic and hyperopic patients. There are several potential complications that may have hindered the widespread adoption of this procedure. These complications include elevation of intraocular pressure (IOP), cataract induction, and iris pigment dispersion. These issues are particularly associated with lens designs that are permanently fixed in the eye by attachment to anatomical structures such as the ciliary sulcus and iris.

Floating phakic refractive lenses may preserve eye dynamics and significantly reduce the risks of complications associated with earlier phakic refractive lens designs. The floating design facilitates aqueous humor flow within the eye, thereby reducing or eliminating the risk of intraocular pressure (IOP) elevation. It also may reduce or minimize the likelihood of contact between the refractive lens and the natural crystalline lens, which could induce cataracts, or forced connections to the iris, which can lead to iris pigment dispersion. The floating lens design addresses these important issues by accommodating the dynamic changes within the eye, such as those occurring during accommodation. However, this freedom of movement introduces the potential for decentration, where the lens may shift away from the optical center of the eye within the iris opening (pupil). Decentration could result in a rare but potentially severe complication: migration of the lens past the zonules and into the vitreous cavity behind the natural crystalline lens. The zonules are delicate fibers that connect the ciliary processes of the eye to the natural crystalline lens. In individuals with very high degrees of myopia or hyperopia, the zonules may be weakened or even detached. If one side of a decentered floating phakic refractive lens, such as the tip of the haptic member, comes to rest on the zonules, further loss of zonular integrity could allow the lens to slip through the gap. In such cases, additional surgical intervention may be need to retrieve the phakic refractive lens.

Astigmatism is a common vision condition that occurs when the eye's cornea or lens has an irregular shape, causing light to focus improperly on the retina. This results in blurred or distorted vision at various distances, often accompanied by eye strain and headaches. A significant portion of the population experiences mild to high levels of astigmatism, with its prevalence increasing as people age. For those with moderate to high astigmatism, daily life can become challenging without proper correction. Toric patients, in particular, face unique difficulties when their astigmatism is not fully addressed. Simple tasks like reading, driving, or using digital devices can become frustrating and tiring. The struggle to focus clearly can lead to decreased productivity at work or school, and may even limit participation in certain activities or hobbies. Over time, this persistent visual impairment can take a toll on an individual's overall well-being, potentially leading to reduced confidence, increased stress, and a diminished quality of life. By contrast, when astigmatism is properly corrected, patients often report a dramatic improvement in their visual comfort and daily functioning, highlighting the importance of accurate and comprehensive vision care.

### SUMMARY

Including a toric surface in an intraocular lens for providing astigmatic correction, however, can be challenging when the intraocular lens is floating lens. Movement of the intraocular lens such as for example rotation of the lens may cause the different power axes to become misaligned within the eye. As discussed herein, different design features may be incorporated into the phakic refractive intraocular lens to address such issues.

Various implementations comprise, for example, a phakic refractive lens comprising a lens body having first and second longitudinally spaced ends and first and second laterally spaced edges. The lens body has anterior and posterior sides and a thickness therebetween. The lens body is curved such that the posterior side is concave. The body comprises material that is buoyant in aqueous humor. An optical zone is centrally located within the lens body. The optical zone is transparent and curved so as to refract light incident on the anterior side thereof thereby providing refractive optical power. The optical zone has a non-rotationally symmetric (or rotationally asymmetric) anterior surface with different first and second curvatures along different first and second radial directions such that different optical power is provided along different directions. A hole extends from an anterior side to a posterior side of the phakic refractive lens. At least one haptic extends to the first and second ends of the lens body. The haptic includes first and second rounded lobes on each of the first and second longitudinally spaced ends. The lens body has a lateral width that is narrower closer to the optical zone than at the widest lateral separation between the rounded lobes at the first end. The first and second laterally spaced edges have reduced distance therebetween at the center of the phakic refractive lens and increased distance therebetween at the widest separation of the laterally spaced edge at the first end.

In some implementations, a phakic refractive lens has first and second longitudinally spaced ends and first and second laterally spaced edges. The phakic refractive lens has first and second sides and a thickness therebetween. The phakic refractive lens comprises an optical zone, a hole configured to allow aqueous humor to flow therethrough between first and second sides of the phakic refractive lens, and a support extending to the first end and/or the second end of the phakic refractive lens, respectively. The optical zone is centrally located between the first and second longitudinally spaced ends. The optical zone is transparent and curved so as to refract light incident on the first side thereof thereby providing refractive optical power. The phakic refractive lens comprises material that is buoyant in aqueous humor. Additionally the phakic refractive lens has a lateral width that is narrower closer to the optical zone and wider farther from the optical zone and closer to at least one of the first and second longitudinally spaced ends.

In some implementations, a phakic refractive lens has first and second longitudinally spaced ends and first and second laterally spaced edges, as well as first and second sides and a thickness therebetween. The phakic refractive lens comprises an optical zone, a hole configured to allow aqueous humor to flow therethrough between first and second sides of the phakic refractive lens, and a support extending to the first end and/or the second end of the phakic refractive lens, respectively. The support includes first and second rounded lobes at first and second corners at one or both of the first and second ends. The optical zone is centrally located between the first and second longitudinally spaced ends. The optical zone is transparent and curved so as to refract light incident on the first side thereof thereby providing refractive optical power. The optical zone has a non-rotationally symmetric (or rotationally asymmetric) first surface with different first and second curvatures along different first and second radial directions such that different optical power is provided along different directions. Additionally, the phakic refractive lens comprises material that is buoyant in aqueous humor.

### BRIEF DESCRIPTION OF THE DRAWINGS

The drawings described herein are for illustrative purposes only of selected embodiments and not all possible implementations, and are not intended to limit the scope of the present disclosure.

Corresponding reference numerals indicate corresponding parts throughout the several views of the drawings.

FIG. 1 is a cut-away view of an eye showing the positioning of an example phakic lens in the posterior chamber.

FIG. 2 is a top or front view of an example intraocular lens (e.g., a toric lens).

FIG. 3 is a sideview of an example intraocular lens (e.g., a toric lens).

FIGS. 4A and 4B are top or front views of an example lens (e.g., a toric lens) including holes therein, for example, in the optical zone and in the rounded lobes of the haptic(s).

FIG. 4C is a top or front view of the design shown in FIGS. 4A and 4B with various angles and curvatures identified.

FIG. 5A is a cross-sectional view of the example lens (e.g., a toric lens) of FIG. 4A through the line A-A showing dimensions (e.g., length and thickness) of some features.

FIGS. 5B and 5C are enlarged cross-sectional views through portions of the cross-section shown in FIG. 5A identified as B and C.

FIG. 6A is a perspective view of the intraocular lens of FIGS. 4A-4C as well as 5A-5C showing the concave posterior side.

FIG. 6B is a perspective view of the intraocular lens of FIGS. 4A-4C as well as 5A-5C showing the convex anterior side.

### DETAILED DESCRIPTION

Various posterior chamber floating phakic refractive lenses described herein may incorporate specific design features and materials, that may be implanted in the posterior chamber of the human eye to address refractive errors. Such lenses may be designed to float in the aqueous humor and may be constructed to be flexible and soft. The floating design can include one or more of a thin, rectangular-like buoyant lens body, curved concave edges, rounded lobes in the corners, and indentations therebetween. The floating design may preserve eye dynamics and reduce the risks of complications associated with phakic refractive lens implantation, such as cataract induction of the natural crystalline lens, iris pigment dispersion, and intraocular pressure elevation.

Various designs described herein incorporates a small central hole in the lens optic, allowing aqueous humor to flow through. This flow is intended to exert a centering force on the floating phakic refractive lens while maintaining its optical function. The floating lens design is structured to utilize this aqueous flow to promote stable centration of the optic and assist in positioning the optic body within the pupillary space. The floating lens design may maintain a gap between the phakic refractive lens and the natural crystalline lens or may configure the lens to have reduced or only minimal contact with, e.g., to not adhere to, the natural crystalline lens.

As referenced above, the phakic refractive lenses may also include concave laterally disposed edges such that the lens extends inward in the lateral direction proximal to the center of the lens, which may permit flow of aqueous and/or permit more conformal integration with the internal structures of the eye.

In various implementations, the corners of the lens are designed as rounded lobes, functioning as gentle anchor points within the sulcus angle. The lobes enable secure, non-rigid anchoring within the sulcus angle while allowing controlled axial displacement providing stability without rigid fixation, thereby reducing the risk of tissue irritation or damage. They contribute to overall lens stability by preventing unwanted rotation or lateral movement while allowing for controlled axial displacement. Toric lens configurations described herein can thus provide precise rotational stability, beneficial for effective astigmatic correction.

Constructed from biocompatible materials with a specific gravity approximating the aqueous medium, the lens achieves improved buoyancy and dynamic interaction with ocular physiology. As discussed above, various lens designs incorporate geometric enhancements, such as concave laterally disposed edges (possibly varying radii of curvature) and/or finely tuned angular parameters, to provide seamless integration with the eye's anatomy. The phakic refractive lenses, such as the toric lenses, described herein may provide a safe, reversible, and effective solution for patients with complex refractive needs, offering superior optical performance, long-term stability, and enhanced visual outcomes while reducing surgical risks.

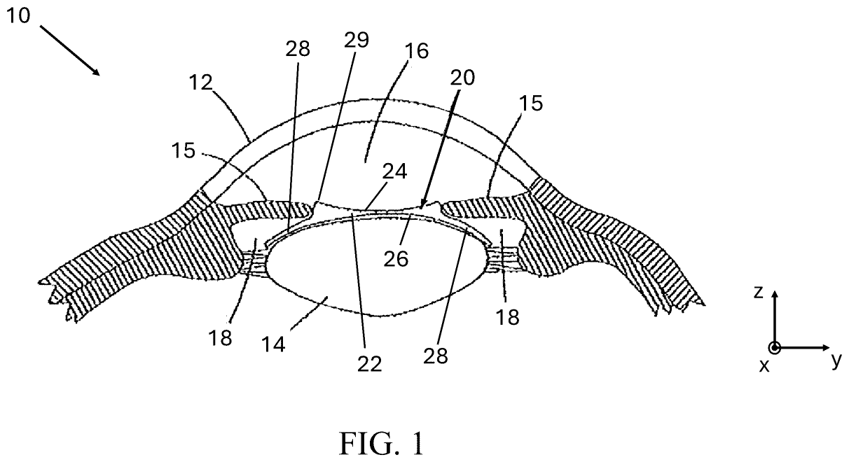

As is well known, an eye 10, a rendering of which is shown in FIG. 1, has a cornea 12, lens 14 (often referred to as the natural or crystalline lens), and an iris 15. An anterior chamber 16 is between the cornea 12 and the iris 15 and lens 14, while a posterior chamber 18 is just behind the iris 15.

Various phakic refractive intraocular lenses 20 disclosed herein can be inserted into the posterior chamber 18 of the eye 10. FIG. 1 schematically illustrates an example of a phakic refractive intraocular lens 20 inside the posterior chamber 18 of the eye 10.

The phakic refractive lens 20 has an optical zone 22 or optic centrally located on the lens. The optical zone, optic, or optical body 22 is optically transparent and may comprise one or more curved surfaces (e.g., a curved anterior surface 24 and/or curved posterior surface 26) that refracts light to provide optical power to the optical zone or optical body. Light transmitted through the cornea 12 passing through the optical zone 22 may thereby be refracted prior to reaching the natural lens 14 of the eye. The optical power of the optical zone 22 may supplement the optical power of the natural lens 14 and cornea 12 of the eye 10 to provide refractive correction. In various phakic refractive lenses 20 disclosed herein, the optical zone or lens optic 22 has a non-rotationally symmetric surface (or rotationally asymmetric), e.g., anterior surface 24, with different first and second curvatures along different first and second radial directions such that different optical power is provided along different directions. This surface 24 may, for example, be a toric or toroidal surface. This surface 24 may in some cases provide correction for astigmatism in the eye 10.

The phakic refractive intraocular lens 10 may also include a support 28 such as one or more haptics that assist in stabilizing placement of the lens in the eye 10. This support 28 may extend from the optical zone or optical body 22, possibly with a transition region therebetween. In the example shown in FIG. 1, a protrusion or ridge 29 at the perimeter of the optical zone 22 may assist in centering (e.g., laterally) the phakic refractive lens 20 within the eye. The iris 15 may, for example, push up against the protrusion or ridge 29 at times when the iris is sufficiently constricted.

Various phakic refractive intraocular lenses 20 described herein comprise floating phakic refractive lenses. The phakic refractive floating lens 20 may, for example, comprise material having a density such that the phakic refractive lens is buoyant in the aqueous humor in the eye 10. In various implementations the phakic refractive lens 20 exhibits neutral buoyancy or is within ±10% or ±5% thereof. The shape and/or material of the phakic refractive lens 20 may contribute this buoyancy and/or floating behavior.

Moreover various designs described herein can enhance floating phakic refractive intraocular lenses 20 by addressing limitations thereof while preserving the floating mechanism's benefits. Various self-centering phakic refractive lenses described herein introduce a floating design that offers significant advantage by preserving eye dynamics and reducing risks associated with fixed lens designs. This floating nature can present limitations, particularly for patients with astigmatism. The intraocular lens 20, designed to float freely in the aqueous humor of the posterior chamber, experiences some degree of rotation and lateral displacement (e.g., displacement in x and/or y direction). While this movement allows the lens 20 to adapt to dynamic changes in the eye, it can lead to decentration away from the optical center within the iris opening and rotate away from proper azimuthal orientation. This inherent movement also poses a challenge for providing stable toric correction, which benefits from precise and consistent alignment and orientation to effectively address astigmatism. Unfortunately, the rotational and lateral movements of the floating lens may likely result in fluctuating vision quality for astigmatic patients. Despite its innovative aspects, a floating phakic lens design may thus limit consistent and effective toric corrections to patients with astigmatism if its inherent mobility within the eye is not adequately controlled or countered. Lens features described herein, however, can address such issues.

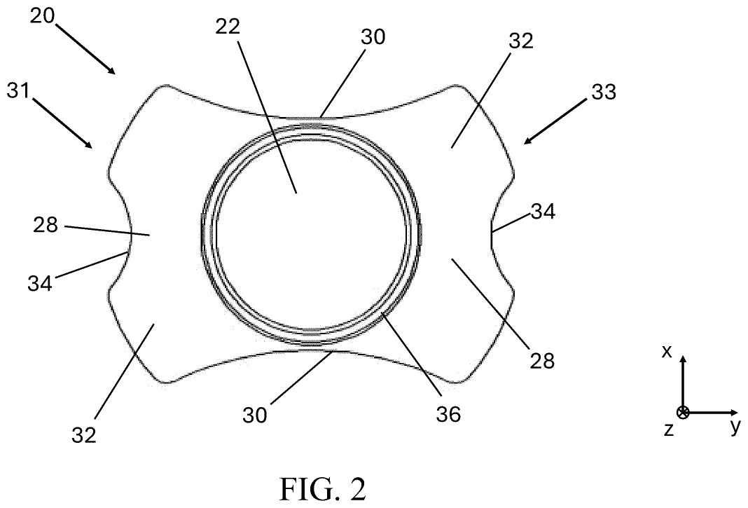

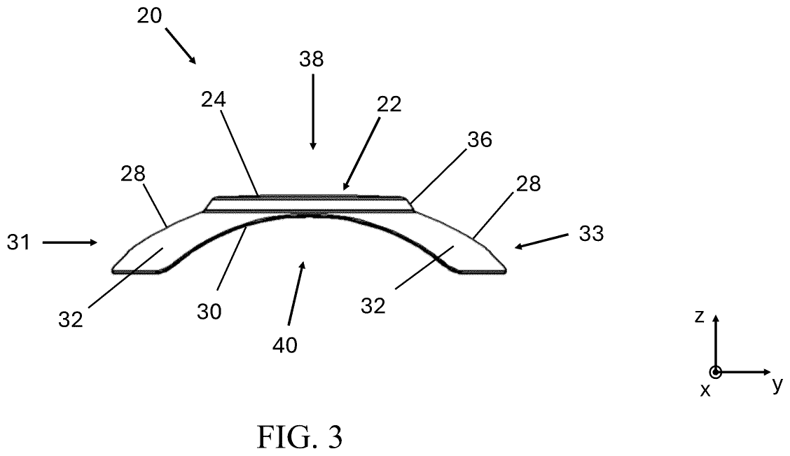

As shown in FIGS. 2 and 3, various intraocular lenses 20 described herein feature a generally rectangular shape with concave edges 30 (e.g., as seen from the front or top view), possible varying radii, and rounded corner lobes 32, potentially resulting in a slightly larger overall size (e.g., as measured along a diagonal from a first lobe on a first end 31 to second lobe on a second end 33 catty-corner to the first lobe). Various features of the designs allow secure anchoring of the haptic(s) 28 within the sulcus angle, reducing or preventing lateral displacement (e.g., in x and/or y direction) and/or azimuthal rotation (e.g., about the optical axis of the optic body). Despite this anchoring, the lens 20 remains flexible and soft, maintaining axial displacement (e.g., in z direction) through aqueous humor flow. This design preserves the floating action, potentially enabling dynamic adaptation to eye movements and/or maintaining a gap with the natural crystalline lens 14. By reducing risks associated with fixed designs (such as for example cataract induction, iris pigment dispersion, and intraocular pressure increases) and combining improved geometry with carefully engineered material properties, various designs described herein offer a stable yet dynamic solution for correcting refractive errors, including astigmatism, while safeguarding eye health and functionality.

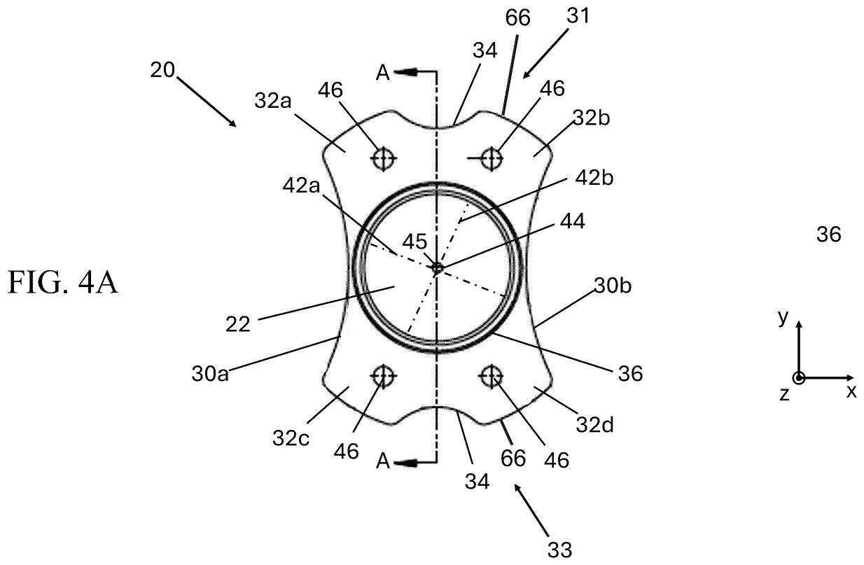

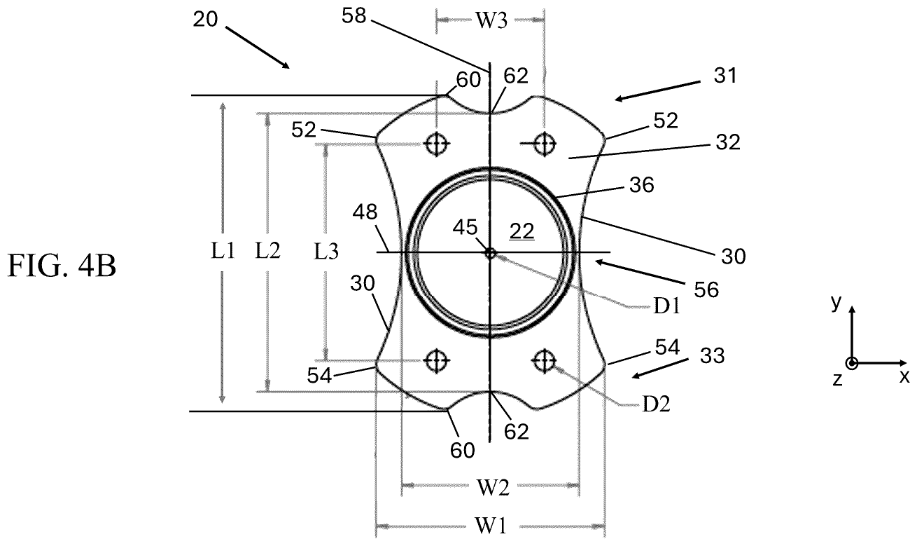

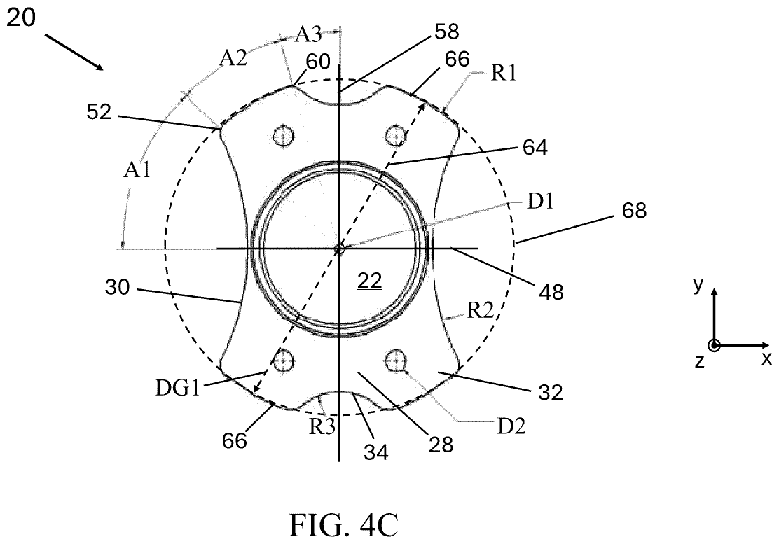

As referenced above, the lenses 20 can feature a rectangular-like overall shape (e.g., as seen from the front or top view) with gently curved edges (e.g., concave edges 30 on the longer sides), indentations 34 on the shorter sides, or both curved edges and indentations. The indentations 34 can be symmetrical about a central plane aligned with the longitudinal direction and/or the longitudinal axis 58 (see, e.g., FIG. 4B) of the lens 20. At the center, there is a circular optical zone or lens body 22 that provides refractive optical power, surrounded by an outer ring transition zone 36. Beyond the transition zone 36, haptic extensions 28 curve outward symmetrically from the central area (e.g., about lateral and/or longitudinal axes), forming a broader, rounded rectangular-like frame (e.g., as seen from the front or top view). The overall design creates a balanced and symmetrical profile (e.g., symmetrical about longitudinal and lateral axes through the center of the lens 20 and the optical zone 22).

As shown in FIG. 3, from the side, the intraocular lens 20 exhibits a thin, convex-concave or concave-convex shape. The lens 20 has first and second (e.g., anterior and posterior) sides 38, 40 and a thickness therebetween. The refractive intraocular lens 20 is curved with the haptic(s) 28 bending with distance away from the optical zone 22 such that the first (e.g., anterior) side 38 is substantially convex. The second, e.g., posterior, side 40 is concave. The second, concave side 40 is configured to face posteriorly (toward the natural crystalline lens 14) when implanted. Being concave, the second side 40 fits more closely with the convex anterior surface of the natural lens 14 of the eye 10, for example, than if the posterior surface were plano or convex.

In various designs, the central optical zone or lens body 22 is slightly thicker in at least portions thereof than the haptic(s) 28 and smoothly transitions into a thinner outer zone of the lens 20 toward the edges in some implementations. As referenced above, the haptic sections 28 extend outward in a slightly curved manner, potentially bending and/or tapering smoothly (e.g., in thickness) in some designs to maintain a streamlined and seamless structure. The entire shape is gentle and fluid, providing smooth continuity between the different sections of the lens 20 and without abrupt changes or sharp features (e.g., steps, ridges, grooves, discontinuities, interruptions or any combination of these) in various implementations. The lens support or haptic(s) 28 extends from the optical zone 22 smoothly and/or without undulation and/or corrugations in various implementations.

In various implementations, the intraocular lens 20 comprises a single monolithic structure and is not made up of separate components, for example, assembled or fit together. The lens 20, for example, may comprise a body comprising a material, the body including both the optical zone 22 and the support 28 extending therefrom, possibly via a transition zone 36. This body may comprise, for example, a soft membrane made of, for example, silicone. The body may be molded so as to form the optical zone 22, support 28, and optional transition region 36 therebetween, integrated together monolithically in the body (e.g., the membrane). The material comprising the body may in various implementations be homogenous in composition throughout the body and thus throughout the lens 20. In other cases, the lens 20 and the support 28 could be formed separately and joined to each other by a suitable technique.

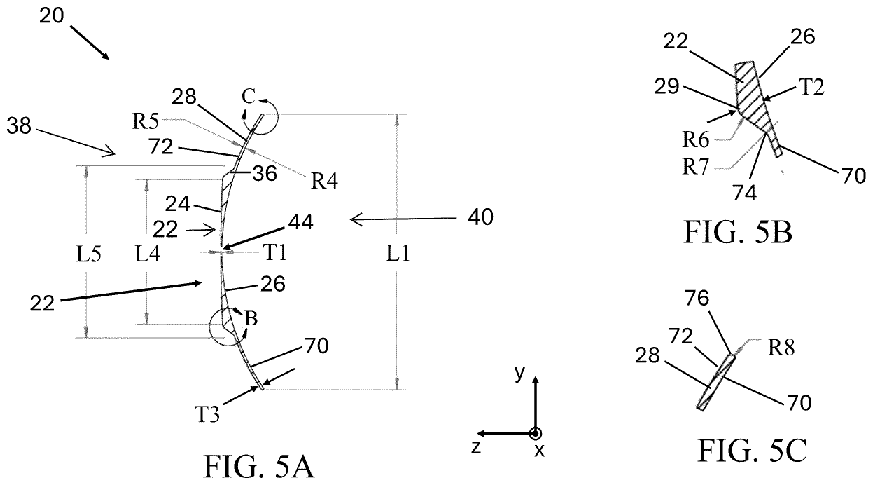

FIG. 4A depicts another lens design. This lens 10 design may include any one or more of the features discussed above with regard to the lenses and/or lens designs shown in FIGS. 1-3. Likewise, any of the features of the lenses and lens designs discussed in connection with FIGS. 4A-6B may be applied to and/or be included in lenses and lens designs such as shown in FIGS. 1-3 and/or discussed with respect thereto. The phakic refractive intraocular lens 20 shown in FIG. 4A comprises a body, e.g., an approximately centimeter squared sized membrane or piece of material with millimeter and/or submillimeter thickness, having first and second longitudinally spaced ends 31, 33 (e.g., separated from each other in the y direction in FIG. 4A) and first and second laterally spaced edges 30a, 30b (e.g., separated from each other in the x direction in FIG. 4A). The lens 20, for example, the body of the lens, has anterior and posterior sides 38, 40 (see FIG. 5A) and a thickness therebetween. As referenced above, this thickness may be about a millimeter or less than a millimeter, e.g., tens or hundreds of micrometers thick. The intraocular lens 20, e.g., the body of the lens, is curved (e.g., in its natural resting position) such that the posterior side 40 is concave. The lens 20, the body of the lens, comprises material that is buoyant in aqueous humor.

An optical zone 22 is shown centrally located within the body of the lens 20. As discussed above, the optical zone 22 is transparent and curved so as to refract light incident on the anterior side 38 thereof (e.g., the anterior surface 24 of the optical body) thereby providing refractive optical power. For example, the anterior surface 24 may have a curvature to provide an appropriate amount of optical power to provide refractive correction for the eye 10. In some cases, the patient may suffer from hyperopia while in other cases, myopia. The curvature of the anterior surface 24 of the optical zone may thus in some designs be convex although in some cases may possibly be concave and may have different amounts of curvature depending on the prescription of the patient. In some cases, for example, where the intraocular lens 20 is designed to provide for astigmatic correction, the optical zone 22 has a non-rotationally symmetric (or rotationally asymmetric) anterior surface 24 with different first and second curvatures along different first and second radial directions 42a, 42b such that different optical power is provided along different directions. In some implementations, this non-rotationally symmetric (or rotationally asymmetric) anterior surface 24 may be a toroidal or toric surface. The different curvature and optical power in different radial directions 42a, 42b may be used to reduce the effect of astigmatism or cylinder of the eye 10. One of the radial directions 42a, 42b may, for example, coincide with the axis of the patient's cylinder prescription. Thus, the radial directions 42a, 42b may vary for different lenses 20 as the prescription of the patients vary. The lens 20 is configured to be placed in a consistent manner. As such, the axes 42a, 42b would be formed at rotational positions for a particular patient. In other cases, the axes 42a, 42b are formed in a common location amongst a kit of lenses and the lens 20 is placed in the proper rotational position for the particular patient during implantation.

In the design shown in FIG. 4A, the intraocular lens 10, e.g., the body of the lens, includes at least one hole, here a plurality of holes, extending from the anterior side 38 to the posterior side 40 of the phakic refractive lens. In this example, such a hole 44 is included in the optical zone 22 and, in particular in this design, at the center 45 of the optical zone 22 (which in this case is circular as seen from the top view). In this design, additional holes 46 are included in the support or haptic(s) 28, for example, in the rounded lobes 32 of the haptic(s) or support. The holes 44, 46 may in various implementations permit the flow of aqueous humor therethrough. In the example shown, a single hole 44 is included in the optical zone 22. In various designs only a single hole 44 is included in the optical zone. In other designs, only peripheral holes 46 are provided, e.g., one, two, three, or four, or more than four peripheral holes 46 and the central area of the optical zone 22 is continuous, e.g., without the hole 44. In the lens 20 shown, four holes are included toward, in the direction of or actually in the four rounded lobes 32 of the support or haptic(s) 28. This number may be larger or smaller in different designs. Likewise not all four holes may be included and/or their placement may be different from that illustrated. Some lobes 32 may have holes 46, others may not, or all may have holes or none of the lobes may have holes. On a given lens 20, the placement of the holes 46 may be at different radial distances from the center of the optical zone 22.

As referenced above the intraocular lens 20 includes at least one support 28 extending to the first and/or second ends 31, 33 of the lens 20 or lens body. The support 28 may be referred to herein as a haptic. As discussed above, the support or haptic(s) 28 may include rounded lobes 32 at the ends 31, 33 of the lens or body or haptic(s). In this example, the haptic 28 includes first and second rounded lobes 32a, 32b at first and second corners of the first end 31 as well as first and second rounded lobes 32c, 32d at first and second corners of the second end 33. In the example shown, four lobes 32a, 32b, 32c, 32d are shown. The rounded lobes 32 have rounded distal edges 66 that may reduce injury to the inner structure of the eye 10. The rounded lobes 32 may extend into the sulcus angle increasing the stability of the lens position and/or orientation without damaging the internal physical structure of the eye 10.

Between the rounded lobes 32 are indentations 34. For example, on the first end 31 of the intraocular lens 20 or lens body is a pair of rounded lobes 32a, 32b and an indentation 34 therebetween. Similarly, on the second end 33 of the lens 20 or lens body is a pair of rounded lobes 32c, 32d and an indentation 34 therebetween. As shown the indentation 34 may be curved, for example, may have a concave curvature. The indentation 34 may provide a pathway for aqueous fluid to flow when the rounded lobes 32 are in the sulcus of the eye 10. The indentations 34 may also allow some tissue in the sulcus angle to come to rest between the adjacent lobes 32. The ingress of this tissue may be slight to not prevent the floating behavior described herein but may be sufficient to provide resistance to rotation to the lens 20. Providing a resistance to rotation can be important to the extent that the optical zone 22 has a non-rotationally symmetric (or rotationally asymmetric) power, e.g., toric configuration, as discussed above.

The lens perimeter features concave curved (e.g., laterally disposed) edges 30 (which may have a single radius of curvature as seen from the top or front view and possibly with varying radii of curvature from the perspective view), enabling it to conform more naturally to the eye's internal structures, particularly the sulcus angle. The concave design also interacts with the flow of aqueous humor, helping to maintain the lens's position and supporting its dynamic behavior within the eye 10. Although curvature on the lateral edges 30 is less than the curvature of the indentations 34, some degree of tissue interaction along the concave curve, e.g., between the lobes 32a, 32c and/or between the lobes 32b, 32d can aid in resisting undesirable rotation of the lens 20 relative to the eye.

Likewise, as shown in FIG. 4B, the lens 20 is not widest at the center 45 (e.g., at the laterally directed axis 48) at the center. Nor does the lens 20 protrude outward laterally (e.g., in the +x and/or −x direction), for example, at the center. In this design, the lateral width of the lens 20 (e.g., in the x direction along the direction of the laterally directed axis 48 through the center 45 of the lens 10) is narrower closer to the optical zone 22 than at the widest part of the lens, which is at the lobes 32. The first and second laterally spaced edges 30a, 30b, for example, have reduced distance therebetween at the center 45 of the phakic refractive lens 20 (e.g., at the laterally directed axis 48 through the center of the optical zone 22), width W2, and increased distance therebetween at the widest separation between the pair rounded lobes 32a, 32b at the first end 31 (e.g., at the widest separation between the outer perimeter of the rounded lobes 32a, 32b at the first end 31) and/or at the widest separation between the pair rounded lobes 32c, 32d at the second end 32 (e.g., at the widest separation between the outer perimeter of the rounded lobes 32c, 32d at the second end 32), width W1. Similarly, the first and second laterally spaced edges 30a, 30b have reduced distance therebetween at the center 45 of the phakic refractive lens 20 (e.g., at the lateral directed axis 48 through the center of the optical zone 22), width W2, and increased distance therebetween at the widest part of the lens or the farthest most locations 52 on the perimeter of the lens in the lateral or x directions at the first end and/or at the widest part of the lens or the farthest most locations 54 on the perimeter of the lens in the lateral or x directions at the second end, width W1. This feature is a result of the concave edges 30, which cause the lens 10 to decrease in width along, e.g., the laterally directed axis 48, as the perimeter approaches the center 45 of the lens. As shown, W2 is smaller than W1. The middle 56 of the lens 20 (where the optical zone 22 is located and more particularly, where the center 45 of the lens and/or of the optical zone 22 is located) is less wide than the widest part of the lens, which is closer to the first and/or second ends 31, 33. The lens 20 is thinner in the middle is a result of the concave lateral edges 30. The width of the lens 20, W, along the lateral direction (the shorter direction) increases from the center 45 of the lens 20 toward the rounded lobes 32.

In some implementations, the width, W2, of the lens 10 at the center 45 (e.g., in the x direction or along the direction of the lateral axis 48), may be at least 4 mm, 5 mm, 6 mm, 7 mm, 8 mm, 9 mm, 10 mm, 11 mm, 11.5 mm or any range formed by any of these values or possible larger or smaller, for example, in a range from 6 mm to 8 mm, e.g., 7.24 mm. The width W1, of the lens 10 at the widest part thereof (e.g., in the x direction or along the direction of the lateral axis 48), for example, between the points 52 at the first end or the points 54 at the second end, may be at least 5 mm, 6 mm, 7 mm, 8 mm, 9 mm, 10 mm, 11 mm, 12 mm, or any range formed by any of these values or possible larger or smaller, for example, in a range from 8 mm to 10 mm, e.g., 9.3 mm.

In some implementations, the length, L1, of the lens 20 in the longitudinal direction, e.g., along the longitudinal axis 58, may be at least 10.0 mm, 11.0 mm, 11.7 mm, 11.8 mm, 11.9 mm, 12.0 mm, 12.1 mm, 12.2 mm, 12.3 mm, 12.4 mm, 12.5 mm, 12.6 mm, 12.7 mm, 12.8 mm, 12.9 mm, 13.0 mm, 13.1 mm, 13.2 mm, 13.3 mm, 13.4 mm, 13.5 mm, 13.6 mm, 13.7 mm, 14.0 mm, 15.0 mm, or any range formed by any of these values or possibly larger or smaller, for example in a range from 11.7 mm to 13.7 mm, e.g., 12.7 mm. This length, L1, is the distance in the longitudinal direction between the points 60 on the lens farthest from each other in the longitudinal direction. As referenced above, the lens 20 may be rectangular like (e.g., as seen from the front or top view). The lens 20 is longer in one direction, e.g., in FIG. 4B, the y direction. This direction is referred to as the longitudinal direction. The longitudinal axis 58 is directed along this longitudinal direction. The lens 20 is shortest along the orthogonal direction, which is often referred to herein as the lateral direction. Similarly, the lens 20 is shortest along the lateral axis 48 directed along this direction. As stated above, the width of the lens 20 is less in the direction along the lateral axis 48, e.g., as compared to the direction along the longitudinal axis 58.

The lens 20, at least from a top or front view, may be symmetrical about the longitudinal axis 58, e.g., a plane therethrough orthogonal to the lateral axis. Moreover, the haptic or haptics 28 may be symmetrical about the longitudinal axis 58, e.g., a plane therethrough orthogonal to the lateral axis. Similarly, the lens 20, at least from the top or front view may be symmetrical about the lateral axis 48, e.g., a plane therethrough orthogonal to the longitudinal axis. Moreover, the haptic or haptics 28 may be symmetrical about the lateral axis 48, e.g., a plane therethrough orthogonal to the longitudinal axis. As seen from the top or front, the lens 20 and/or haptic 28 might not be rotationally symmetric, but may exhibit two-fold symmetric, for example, about the longitudinal and/or lateral axis 58, 48. The symmetry of the lens 20 may also depend on the prescription. To provide astigmatic correction, the anterior surface 24 of the optical zone 22 may have different curvatures in different directions, for example, the anterior surface may be toric or toroidal. (In other designs, to provide astigmatic correction, the posterior surface 26 of the optical zone 22 may have different curvatures in different direction, for example, the posterior surface may be toric or toroidal.) The lens 20 may not be symmetrical about the longitudinal and/or lateral axes 58, 48 in such cases depending on the cylinder axis. However, for non-astigmatic correction, such as myopia or hyperopia correction without astigmatism, the anterior surface 24 (and possibly posterior surface 26) may be rotationally symmetric. As a result, the lens 20 may be symmetric (e.g., exhibit two-fold symmetry) about the longitudinal axis 58, e.g., a plane therethrough orthogonal to the longitudinal axis and/or about the lateral axis 48, e.g., a plane therethrough orthogonal to the longitudinal axis (except possibly for the optical zone 22, which may be toric or otherwise not rotationally symmetric or rotationally asymmetric on one, e.g., anterior or posterior, or on both sides and may have a cylinder axis not aligned with the longitudinal and/or lateral axis).

In various designs, the longitudinal axis 58 may bisect the lens 20 into two halves at least as seen from the front or top view. Accordingly, the longitudinal axis 58 may be referred to as the longitudinal bisector in such cases. Similarly, in various designs the lateral axis 48 may bisect the lens 20 into two halves at least as seen from the front or top view. Likewise, the lateral axis 48 may be referred to as the lateral bisector in such cases.

FIG. 4B also shows the length of the lens, L2, along the longitudinal axis 58 that is the shortest. This length, L2, is at the indentations 34 between the rounded lobes 32. The length of the lens 20 is the shortest because of the concave indentations 34. The perimeter of the lens 20 extends inward from the rounded lobes 32 toward the optical zone 22, reducing the length of the lens 10 and support or haptic(s) 28 in those areas. This length, L2, is the distance in the longitudinal direction (e.g., parallel to the longitudinal axis 58) between the points 62 on the respective indentations 34 where the indentation extends inward the most into the lens 20 and/or into the support or haptic(s) 28. As a result, this length L2 is smaller than the length of L1 of the lens 20. In some implementations, this length, L2, may be at least 9.0 mm, 10.0 mm, 10.3 mm, 10.4 mm, 10.5 mm, 10.6 mm, 10.7 mm, 10.8 mm, 10.9 mm, 11.0 mm, 11.1 mm, 11.2 mm, 11.3 mm, 11.4 mm, 11.5 mm, 11.6 mm, 11.7 mm, 11.8 mm, 11.9 mm, 12.0 mm, 12.1 mm, 12.2 mm, 12.3 mm, 13.0 mm, 14.0 mm, or any range formed by any of these values or possibly larger or smaller, for example, in the range from 10.3 to 12.3, e.g., 11.3 mm.

FIG. 4B also shows the distance, L3, in the longitudinal direction between one of the holes 46 in the lobes 32 at the first end 31 and one of the holes in the lobes at the second end 33. In some implementations, this distance may be at least 5 mm, 6 mm, 7 mm, 8 mm, 9 mm, 10 mm, 11 mm, 12 mm, or any range formed by any of these values or possibly larger or smaller, for example, from 7 to 10 mm or 7.8 to 9.8 mm, e.g., 8.8 mm. Additionally, FIG. 4B shows the distance, W3, in the lateral direction (e.g., parallel to the x axis or lateral axes 48) between one of the holes 46 in one lobe 32 at the first end 31 and one of the holes 46 in another lobe at the first end. This distance, W3, may be at least 2 mm, 3 mm, 4 mm, 5 mm, 6 mm, 7 mm, 8 mm, or any range formed by any of these values or possibly larger or smaller, for example, from 3 mm to 6 mm or 3.4 to 5.4 mm, e.g., 4.4 mm. In some designs, this distance, W3, is the same as the distance in the lateral direction (e.g., parallel to the x axis or lateral axes 48) between one of the holes 46 in one of the lobes 32 at the second end 33 and one of the holes in another lobes at the second end. Such would be the case if the lens 20 were symmetric about the lateral axis 48.

In various implementations, the holes 46 are a distance from the perimeter of the lens 20 and/or a perimeter of the support or haptic(s) 28 such as the distal end 66 of the rounded lobe 32 such that internal structures in the eye 10, e.g., zonules, portions of the ciliary body, etc. do not affix to the lens or support/haptic(s) through the hole. In the lens 10 shown, for example, the holes 46 may be at least 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 3.0 mm, 3.5 mm, 4.0 mm from the closest perimeter of the lens 20 or support/haptic(s) such as the distal end 66 of the rounded lobe 32 or any range formed by any of these values (e.g., from 1.2 to 1.9 mm or 1.4 to 1.8 mm) or possible larger or smaller.

As discussed above, in some implementations, the phakic refractive lens 20 may include one or more holes strategically positioned to enhance its performance. In various designs, for example, the phakic refractive lens 20 comprises a hole 44 located in the center of the optical zone 22, with lateral extent such as a diameter, D1, at least 0.05 mm, 0.1 mm, 0.2 mm, 0.3 mm, 0.4 mm, 0.5 mm. 0.6 mm, 0.7 mm, 0.8 mm, 0.9 mm, 1 mm, or any range formed by any of these values, for example, from 0.2 mm to 0.5 mm or 0.3 mm to 0.5 mm, such as 0.4 mm. This central hole configuration allows aqueous humor to flow through the optical body 22, creating a centering force while maintaining optical functionality.

In some designs, the refractive intraocular lens 20 may alternatively or additionally incorporate holes 46 positioned within the haptic(s) 28, for example, in the rounded lobes 32 at the corners of the lens. In various designs, the lateral extent such as diameter, D2, of the holes 46 in the lobes 32 may be at least 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, or any range formed by any of these values or possibly larger or smaller, for example, in range from 0.4 mm to 1.2 mm or 0.6 to 1.0 mm, e.g., 0.8 mm. These corner holes 46 are designed to work in conjunction with the lens's overall geometry, particularly its concave edges 30 and rounded corner lobes 32. The corner hole configuration maintains the lens's ability to anchor within the sulcus angle while facilitating aqueous flow through the peripheral regions of the lens 20.

The phakic intraocular lens's dimensions may be slightly larger than previous designs, with the distance DG1 along the diagonal 64 from lobe 32 to lobe (shown in FIG. 4C) notably increased. This distance, DG1, measured along the diagonal 64 shown in FIG. 4C between a first lobe 32 at the first end 31 of the lens 10 and a second lobe at the second end 33 catty-corner to the first lobe at the first end may be at least 11 mm, 12 mm, 12.5 mm, 12.6 mm, 12.7 mm, 12.8 mm, 12.9 mm, 13.0 mm, 13.1 mm, 13.2 mm, 13.3 mm, 13.4 mm, 13.5 mm, 13.6 mm, 13.7 mm, 13.8 mm, 13.9 mm, 14.0 mm, 14.1 mm, 14.2 mm, 14.3 mm, 14.4 mm, 14.5 mm, 15 mm, 15.5 mm or any range formed by any of these values or possibly larger or smaller, for example, in a range from 12.8 to 13.8 millimeters (e.g., 13.3 mm), defining the overall lens size. This calculated size adjustment improves the anchoring mechanism by enhancing engagement with the sulcus angle, ensuring stability while preserving the lens's floating properties. Moreover, this distance DG1 is not so large, however, as to pose risk to the internal structure of the eye. For example, if the DG1 is too large, the lens 20 may bow and contact the iris 15 potentially contributing to pigment dispersion and possibly leading to blockage in the trabecular meshwork and elevated IOP or glaucoma. Of course, if the diagonal distance or length, DG1, is too small the intraocular lens 20 may be more inclined to movement, for example, rotation (e.g. azimuthal rotation), which can degrade proper astigmatic correction provided by toric lenses.

Also as illustrated in FIG. 4C, in some implementations, the curvature of the end 66, e.g., distal end, of one or more rounded lobes 32 as view from the anterior side 38 of the lens (e.g., top or front view) is determined by the primary radius, R1, which is half of the distance DG1. Accordingly, the radius R1 may be at least 3.5 mm, 4.0 mm, 4.5 mm, 5.0 mm, 5.5 mm, 6.0 mm, 6.1 mm, 6.2 mm, 6.3 mm, 6.4 mm, 6.5 mm, 6.6 mm, 6.7 mm, 6.8 mm, 6.9 mm, 7.0 mm, 7.1 mm, 7.2 mm, 7.3 mm, 7.4 mm, 7.5 mm, 7.6 mm, 7.7 mm, 7.8 mm, 7.9 mm, 8.0 mm, 8.1 mm, 8.2 mm, 8.3 mm, 8.4 mm, 8.5 mm, 9.0 mm, 9.5 mm, 10.0 mm, or any range formed by any of these values or possibly larger or smaller, for example, in a range from 6.4 to 6.9 mm, e.g., 6.65 mm. This large diameter, DG1, and associated radius, R1, provides a rounded end 66 of the lobe 32 that changes, e.g., bends, gradually rather than including sharp points or edges that could damage internal structure of the eye 20 such as in the sulcus. As shown, the end 66 of the lobe 32 follows the path of a circle 68 defined by the diameter, DG1, and radius, R1. This circle 68 and accordingly the radius R1 and diameter DG1 determine the curvature of the end 66 (e.g., distal end) of the lobes 32 of the intraocular lens 20 shown in FIG. 4C.

The secondary radius (R2), also shown in FIG. 4C, determines the concave curvature of the lateral edge 30. In various implementations, the radius, R2, of the lateral edge 30 as seen from the anterior side 38 of the lens 20 (e.g., from a top or front view) may be at least 5 mm, 6 mm, 7 mm, 8 mm, 9 mm, 10 mm, 11 mm, 12 mm, 13 mm, 14 mm, 15 mm, or any range formed by any of these values or possibly larger or smaller, for example, in the range from 8 mm to 12 mm, e.g., 10 mm. This radius, R2, thus establishes the intermediate curve of the edges 30 including those proximal to the optical zone 22. As seen from the perspective view, the curvature of the edge 30 may have multiple radii.

FIG. 4C also shows the radius (R3) of the indentations as seen from the anterior side 38 of the lens 20 (e.g., from a top or front view), which has a circular curvature. The radius of the indentations may be at least 0.5 mm, 1.0 mm, 1.5 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.5 mm, 4.0 mm, 5.0 mm or any range formed by any of these values or possibly larger or smaller, for example, a range from 2 mm to 3 mm, e.g., 2.4 mm. The depth of the indentations (e.g., measured along the longitudinal direction, for example, along the direction of the longitudinal axis 58 or y axis) measured from the start of the indentation or at the most distal end of the indentation, and possibly of the lens, (e.g., corners of the lobe or points 60) on opposite sides of the indentation on a given end (e.g., first end 31 or second end 33), to the base of the indentation at (e.g., point 62) may be at least 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.8 mm, 2.0 mm or any range formed by any of these values such as from 0.5 to 0.9 mm or 0.6 mm to 0.8 mm, e.g., 0.7 mm or possibly larger or smaller. The width of the indentations at the widest part (e.g., measured along the lateral direction, for example, along the direction of the lateral axis 48 or x axis) of the indentation such as measured at the start of the indentation, or at the most distal end of the indentations, or possibly of the lens, (corners of the lobe or points 60) on opposite sides of the indentation on a given end (e.g., first end 31 or second end 33), may be less than 6.0 mm, 5.75 mm, 5.5 mm, 5.25 mm, 5.0 mm, 4.75 mm, 4.5 mm, 4.25 mm, 4.0 mm, 3.75 mm, 3.5 mm, 3.25 mm, 3.0 mm, 2.75 mm, 2.5 mm, 2.25 mm, 2.0 mm, 1.75 mm, 1.5 mm, or any range formed by any of these values such as from 2.75 to 4.75 mm, e.g., 3.75 mm or possibly larger or smaller.

These radii, R1, R2, R3 may thus be considered to be in a hierarchical relationship with R1> R2>R3. In some embodiment, the radii have a hierarchical relationship with R2>R1> R3. Other arrangements are possible.

In various implementations, the perimeter of the lens maintains continuous curvature (e.g., without discontinuities, sharp points or features, etc.) throughout most or all transitions between these parameters, providing smooth integration of all geometric elements. This continuity can provide for proper interaction with ocular tissues and improved lens performance.

The angular relationships between different sections of the lens 20 are also shown in FIG. 4C following an ascending (or descending) pattern. The primary angle (A1) extends from the lateral axis 48 to the lateral most (e.g., farthest in a direction parallel to the lateral axis or the x axis) corner 52 of the rounded lobe 32 as seen from the anterior side 38 of the lens 20 (e.g., top or front view). This angle, A1, measures at least 20, 25, 30, 35, 40 45, 50, 55, 60, 65, 70, 75 degrees or any range formed by any of these values, for example, ranges from 35 to 55 degrees and establishes the main contour of the lens 20, the lateral edge 30 of the lens. If this angle, A1, is too large, the cavity of the edge 30 may be reduced. Having a concave edge 30 may provide a region through which aqueous may flow. However, a sufficiently large angle, A1, provides a sufficiently large concave edge 30 extending across much of the lens 20 and a gentler curvature.

The secondary angle (A2) provides the width of the of the round lobe 32, for example, as seen from the anterior side 38 (e.g., top or front view) of the lens 20. This angle (A2) extends from the lateral most (e.g., farthest in a direction parallel to the lateral axis or the x axis) corner 52 of the rounded lobe 32 to the corner 60 of the rounded lobe closes to the longitudinal axis 58 as seen from the anterior side 38 of the lens 20 (e.g., top or front view). Too small of an angle A2 may, for example, create a sharper lobe 32 while a larger angle and a wider lobe may be more blunt thus reducing damage to tissue in the sulcus. If, however, the angle A2 is too large, the lens 20 may also be difficult to roll for insertion into the eye 10. The size of the angle A2 will also affect the other angles, A1 and A3. In various implementations, this angle A2, measures at least 15, 20, 25, 30, 35, 40, 45 degrees or any range formed by any of these values for example from 25 and 35 degrees, which may create a gentler transition. In various implementations one of the lobes comprises a percentage of the first (or second) end 31, 33 of the haptic such as at least 15%, 20%, 25%, 30%, 35%, 40%, 45% or any range formed by any of these values.

The tertiary angle (A3) determines the width of the indentation 34. This angle, A3, extends from the corner 60 of the rounded lobe 32 closest to the longitudinal axis 58 to the longitudinal axis as seen from the anterior side 38 of the lens 20 (e.g., top or front view). In some implementations, the third angle, A3, is not more than 30, 25, 20, 15, 10, 5 degrees or any range formed by any of these values such as from 10 to 20 degrees, providing the final subtle curve that is sufficient to allow flow of aqueous therethrough. In various implementations, the indentation 34 comprises a percentage, but not too much, of the first (or second) end 31, 33 such as less than 35%, 30%, 25%, 20%, 15%, 10%, or any range formed by any of these values such as 20% to 35% or 15% to 30% or possibly larger or smaller.

In various implementations, A1 is larger than A2 or A3. In some designs, A2 is larger than two times A3 (e.g., A2>2×A3). The width of the indentation 34, as illustrated by the angular width that is obtained by doubling A3 in this example (e.g., 2×A3), may be smaller than the width (e.g., the angular width A2) of the rounded lobe 32. However other designs are possible.

The thickness parameters are carefully controlled. As illustrated in FIG. 5A, the thickness of the optical zone 22 may vary as the optical zone includes an anterior surface 24 configured to provide suitable optical power for the desired prescription to provide correction for myopia or hyperopia and/or astigmatism. In the phakic refractive intraocular lens 20 shown, the thickness at the center of the optical zone 22, T1, is smaller than the thickness farther from the center and the thickness, T2, at the perimeter of the optical zone 22. The central thickness (T1), for example, at the center of the optical zone may be at least or smaller than 0.01 mm, 0.002 mm, 0.025 mm, 0.03 mm, 0.035 mm, 0.04 mm, 0.045 mm, 0.05 mm, 0.055 mm, 0.06 mm, 0.065 mm, 0.07 mm, 0.075 mm, 0.08 mm, 0.09 mm, 0.1 mm, or any range formed by any of these values or possibly larger or smaller, for example, a range from 0.025 mm to 0.075 mm, e.g., 0.050 mm. The thickness, T2, at the perimeter of the optical zone 22 may be at least or smaller than 0.05 mm, 0.1 mm, 0.2 mm, 0.3 mm, 0.4 mm, 0.5 mm, 0.6 mm, 0.7 mm, 0.8 mm, 0.9 mm, 1.0 mm, 1.5 mm, or any range formed by any of these values or possibly larger or smaller, for example, from 0.1 mm to 0.8 mm, e.g., 0.4 mm, in certain implementations. The thickness of the haptic or support 28 at the edge or perimeter of the optical zone 22 may be at least or smaller than 0.02 mm, 0.03 mm, 0.04 mm, 0.05 mm, 0.06 mm, 0.07 mm, 0.08 mm, 0.09 mm, 0.1 mm, 0.125 mm, 0.15 mm, 0.2 mm, 0.25 mm, 0.3 mm, 0.35 mm, 0.4 mm, 0.45 mm, 0.5 mm, 0.55 mm or 0.6 mm or any range formed by any of these values or possibly larger or smaller, for example, from 0.075 mm to 0.125 mm, e.g., 0.1 mm, in certain implementations. The peripheral thickness, T3, (see FIG. 5B) at the periphery of the lens 20, for example, at the periphery of the haptic(s) may be at least or smaller than 0.02 mm, 0.03 mm, 0.04 mm, 0.05 mm, 0.06 mm, 0.07 mm, 0.08 mm, 0.09 mm, 0.1 mm, 0.11 mm, 0.12 mm, 0.13 mm, 0.14 mm, 0.15 mm, or any range formed by any of these values or possibly larger or smaller, for example, from 0.04 to 0.1 mm, e.g., 0.07 mm in certain designs. As such, however, the body of the lens 22 is thin, for example, in the millimeter or micron range (e.g., tens or hundreds of microns) and may be described as a membrane in some cases. The thickness of the lens 20, the body of the lens, or the membrane may be at least or smaller than 0.01 mm, 0.05 mm, 0.1 mm, 0.5 mm, 1.0 mm, 1.5 mm, 2.0 mm, 2.5 mm, 3.0 mm, or any range formed by any of these values or possibly larger or smaller, for example, from 0.025 mm to 1 mm or 2 mm and the support or haptic(s) 28 may be at least or smaller than 0.01 mm, 0.02 mm, 0.03 mm, 0.04 mm, 0.05 mm, 0.06 mm, 0.07 mm, 0.08 mm, 0.09 mm, 0.1 mm, 0.11 mm, 0.12 mm, 0.13 mm, 0.14 mm, 0.15 mm, 0.2 mm, 0.3 mm, 0.4 mm or any range formed by any of these values or possibly larger or smaller, for example, a range from 0.02 mm to 0.2 mm or 0.04 mm to 0.15 mm. The thickness, however, may be larger or smaller.

FIG. 5A also shows the size, e.g., the length, L4, in the longitudinal direction (e.g., in y direction or along the longitudinal axes 58) of the optical zone 22. This optical zone 22 is where the light from the cornea propagates through and is refracted and continues onto the natural lens 14 of the eye 10. The optical zone 22 provides optical correction, for example, for myopia or hyperopia and/or astigmatism/cylinder depending on the patient's prescription. The length, L4, in the longitudinal direction may be at least or smaller than 5.0 mm, 5.5 mm, 5.6 mm, 5.7 mm, 5.8 mm, 5.9 mm, 6.0 mm, 6.1 mm, 6.2 mm, 6.3 mm, 6.4 mm, 6.5 mm, 6.6 mm, 6.7 mm, 6.8 mm, 6.9 mm, 7.0 mm, 7.1 mm, 7.2 mm, 7.3 mm, 7.4 mm, 7.5 mm, 7.6 mm, 8.0 mm or any range formed by any of these values or possibly larger or smaller, for example, from 5.6 and 7.6 mm, e.g., 6.6 mm. This length, L4, extends to the perimeter of the optical zone 22 and in the example shown to the start of the transition region or zone 36.

FIG. 5A also shows the size, e.g., the length, L5, in the longitudinal direction (e.g., in y direction or along the longitudinal axes 58) of the optical zone 22 and the transition region 36. The length, L5, in the longitudinal direction may be at least or smaller than 6.0 mm, 6.6 mm, 6.9 mm, 7.0 mm, 7.1 mm, 7.2 mm, 7.3 mm, 7.4 mm, 7.5 mm, 7.6 mm, 7.7 mm, 7.8 mm, 7.9 mm, 8.0 mm, 8.1 mm, 8.2 mm, 8.3 mm, 8.4 mm, 8.5 mm, 8.6 mm, 8.7 mm, 8.8 mm, 8.9 mm, 9.0 mm, 9.5 mm, or any range formed by any of these values or possibly larger or smaller, for example, from 6.9 and 8.9 mm, e.g., 7.9 mm. This length, L5, extends to the perimeter of the transition zone 36 and in the example shown to the start of the support or haptic(s) 28.

In various designs, the curvature of the posterior side 40 of the lens 20 is constant across the posterior side 40 of the lens. As discussed above, the optical zone 22 has a posterior surface 26, and this posterior surface has a radius of curvature, R4. The support or haptic(s) 28 has as a posterior surface 70 with the same radius of curvature, R4, as does the transition region 36. The posterior side 40 of the lens 20, e.g., of the body of the lens, has a single surface 26, 70 across the optical zone 22, support or haptic(s) 28 and optical transition region 36 and this single surface has the same curvature and radius of curvature, R4. As such the posterior side 40 of the lens 20 or lens body has a continuous, seamless surface 26, 70 (e.g., without discontinuities, sharp points or features such as seams, etc.), which can be beneficial for fitting in the eye 10 with the natural crystalline lens 14 adjacent thereto. In various implementations, this posterior side 40 and the posterior surface 26, 70 of the lens 20 or lens body (e.g., membrane) is smooth and without steps, ridges, grooves, undulation, corrugation, discontinuities, sharp points or features, interruptions or any combination of these. This posterior side 40 and the posterior surface 26, 70 of the lens 20 or lens body (e.g., membrane) is concave and can fit conformally with respect to the convex natural lens 14 of the eye. In various implementations, the radius of curvature, R4 of the posterior side 40 of the lens 20 or lens body may be at least or smaller than 7 mm, 8 mm, 9 mm, 10 mm, 11 mm, 12 mm, 13 mm, or any range formed by any of these values or possibly larger or smaller, for example, in a range from 9 mm to 11 mm, e.g., 10 mm.

In various designs, the anterior side 38 of the support or haptic(s) 28 has the same curvature as the posterior side 40. As shown in FIG. 5A, for example, the anterior side 38 of the support or haptic 28 has a radius of curvature, R5. The support or haptic(s) 28 may have an anterior surface 72 and this surface may have the same curvature as the posterior surface 70 of the support or haptic(s). For example, the support or haptic(s) 28 may have an anterior surface 72 with a radius of curvature R5. This radius of curvature, R5, may be at least or smaller than 5 mm, 6 mm, 7 mm, 8 mm, 9 mm, 10 mm, 11 mm, 12 mm, 13 mm, 14 mm, 15 mm, or any range formed by any of these values or possibly larger or smaller, for example from 9 to 11 mm and may, e.g., be 10 mm. Accordingly, in various designs, the radius of curvature, R5, of the anterior side 38 and the anterior surface 72 of the support or haptic 28 may be the same as the radius of curvature, R4, of the posterior side 40 and the posterior surface 70 of the support or haptic(s).

In various implementations, this anterior surface 72 is smooth, seamless, and/or uncorrugated and/or without a step, ridge, groove, discontinuity, interruption, sharp point or features, undulation or any combination of these from the transition 36 to the end of the haptic 28 and/or from or over at least ⅔, ½, ⅓ of the distance from the end of the haptic (e.g., from distal end 66 of the lobe 32) to the transition 36 and/or at least over a distance of 1 mm, 1.5 mm, 2.0 mm, 2.5 mm, 3.5 mm, 4.0 mm, or any range formed by any of these values, for example from 1.5 mm to 3.0 mm or 2.0 mm to 2.5 mm, e.g., 2.4 mm, from the end of the haptic (e.g., from distal end 66 of the lobe 32) to toward transition 36.

An enlarged view of the transition region 36 is shown in FIG. 5B. This transition region or zone 36 starts at the perimeter of the optical zone 22 where the edge 29 of the optical zone in this example protrudes from the anterior side 38 of the body of the lens. In various implementations, this edge, ridge or protrusion 29 is not sharp. This edge, ridge or protrusion 29 may have a radius of curvature, R6, that is at least or less than 0.05 mm, 0.1 mm, 0.2 mm, 0.3 mm, 0.4 mm, 0.5 mm, or any range formed by any of these values or possibly larger or smaller, for example, in the range of 0.1 mm to 0.4 mm, e.g., 0.2 mm in some implementations. The thickness of the lens 20 or lens body may smoothy and/or continuously tapers down to the thickness of the support or haptic 28 in various designs such as the example shown in FIG. 5B. Such smooth and/or continuous transition, without seams, steps, corrugation, undulation, discontinuities, sharp points or features etc, or any combination thereof may be more conducive to including in the eye 10 without damage, e.g., abrasion, injury, wear, etc. to the internal structure of the eye. Similarly, the transition region or zone 36 has a radius of curvature, R7 at the junction 74 between the transition region 36 and the support or haptic(s) 28. This radius of curvature, R7, may be at least 0.05 mm, 0.1 mm, 0.2 mm, 0.3 mm, 0.4 mm, 0.5 mm, or any range formed by any of these values or possibly larger or smaller, for example, a range from 0.1 mm to 0.4 mm, e.g., 0.2 mm, thereby providing a smooth surface feature.

FIG. 5C shows an enlarged view of the end 76 of the support or haptic(s) 28. In addition to not being rigid, this end 76 is not sharp and/or pointed. As illustrated, for example, this end 76 has a radius of curvature, R8, which may be at least 0.005 mm, 0.01 mm, 0.02 mm, 0.03 mm, 0.04 mm, 0.05 mm, 0.06 mm, 0.07 mm, 0.08 mm, or any range formed by any of these values or possibly larger or smaller, for example, a range from 0.01 mm to 0.05 mm, e.g., 0.035 mm. This design may therefore potentially reduce damage, e.g., abrasion, injury, wear, etc. to the internal structure of the eye 10 such as to tissue in the sulcus of the eye.

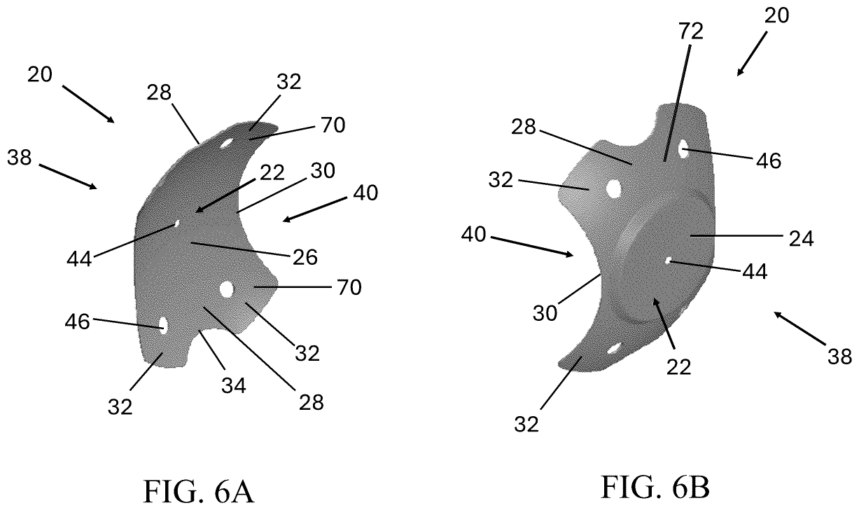

Perspective views of the lens of FIGS. 4A-4C and 5A-5C are shown in FIGS. 6A and 6B. FIG. 6A shows a view of the posterior side 40 of the lens 20 or lens body while FIG. 6B shows a view of the anterior side 38 of the lens 20 or lens body. As shown in FIG. 6A, the posterior side 40 of the lens or lens body comprises a surface 26, 70 that extends across the optical zone 22, the transition zone 36, and the support or haptic(s) 28 that has the same curvature. This posterior surface 26,70 may be spherical. The curvature may be present in the absence of any supporting structure. In other words, the lens 20 is able to sustain this curvature when unsupported. In some cases, the curvature could be responsive to a floating interaction within the eye. Moreover, however, this posterior surface 26, 70 extends smoothly, continuously and/or seamlessly across the optical zone 22, the transition zone 36, and the support or haptic(s) 28 without interruption, without seams, steps, ridges, discontinuities, interruptions, corrugation, undulation, sharp points or features, or any one or more of these, etc, and may be more conducive to including in the eye 10 without damage, e.g., abrasion, injury, wear, etc. to the internal structure of the eye.

Also, as is visible from both FIGS. 6A and 6B, the lens 20 or lens body is curved with the support or haptic(s) 28 extending backward as they increase in distance from the optical zone 22 and/or from the transition region 36. This curve shape is retained by the lens 20 or lens body, while the lens or lens body is in a state with little or no pressure against it (e.g., in the resting state). The lens 20 or lens body can also retain this shape with some pressure against it, however, the material is flexible. For instance, the lens 20 can be rolled up to be inserted through a small incision in the eye.

Various lens designs described herein thus incorporate specific geometric parameters that work in harmony to create an effective and safe phakic intraocular lens.

Moreover, the phakic refractive lens is engineered to function beneficially in the dynamic aqueous environment of the eye. Its material composition is beneficial to its performance, featuring a specific gravity close to that of the surrounding aqueous medium (approximately 0.9 to 1.2 g/cm³). This property enables the lens to achieve buoyancy and float within the eye, much like a parachute in flight. In various implementations the phakic refractive lens 20 exhibits neutral buoyancy in aqueous humor or is within ±30%, ±25%, ±20%, ±15%, ±12%, ±10%, ±8%, ±6%, ±5%, 4%, ±2%, ±1% thereof or any range formed by any of these values. The shape and/or material of the phakic refractive lens may contribute this buoyancy and/or floating behavior. The lens can be fabricated from a range of materials, including hydrophilic or hydrophobic substances, that possess the desired optical properties and foldability with rapid shape recovery. Examples of suitable materials include silicone, silicone polymers, poly(acrylates), poly(methacrylates), hydrogels, proteins, collagens, and their copolymers or mixtures or combinations thereof. These materials typically exhibit a hardness ranging from 20 to 60 Shore A, striking a balance between flexibility and stability. Such material may allow the lens to interact effectively with the natural flow of aqueous humor, which originates from the choroid in the posterior chamber, passes through the pupil, and moves into the anterior chamber. The lens's aspect ratio may be at least 1.2, 1.3, 1.4, 1.5, 1.6, 1.7, 1.8, 1.9, 2.0, 2.1, 2.2, 2.3, 2.4, 2.5, 2.6, 2.7, 2.8, 3.0, or any range formed by any of these values or possibly larger or smaller, ranging from 1.4 to 2.0, and may be comparable to stable and maneuverable parachute designs, improving its performance in the dynamic ocular environment.

The membrane-like flexibility of the lens 20 is a useful feature that enhances its performance and safety within the eye. This innovative design aspect allows the lens 20 to function much like a flexible sail or parachute in wind, responding dynamically to the eye's natural movements and aqueous humor flow. The lens material is engineered to be highly flexible and soft, enabling it to behave more like a thin, pliable membrane or cloak within the eye. This flexibility offers several useful advantages. The lens 20 can readily conform to changes in eye shape during accommodation and other eye movements. This adaptability can enable the lens 20 to maintain adequate positioning and performance despite the eye's dynamic state. The flexible nature of the lens 20 allows it to interact more effectively with the natural flow of aqueous humor. The aqueous humor acts metaphorically as a “wind” that gently pushes against the lens surface, helping to maintain its position and contributing to its dynamic behavior. Despite being anchored at the corners, e.g., providing resistance to unwanted rotation about the central optical axis of the eye 10 when implanted, the central portion of the lens 20 maintains its ability to float freely due to its membrane-like flexibility. This floating action is helpful for adapting to rapid eye movements, maintaining a gap between the phakic lens and the natural crystalline lens, and/or reducing risks associated with fixed designs, such as cataract induction and intraocular pressure increases. The flexibility also allows for better distribution of any mechanical stresses on the lens 20, reducing the risk of localized pressure points that could potentially damage delicate eye structures. Further, the membrane-like nature of the lens 20 enables it to respond more naturally to the eye's accommodation process, potentially offering better visual outcomes across various focusing distances. By combining this membrane-like flexibility with the anchoring mechanism at the corners, the lens achieves a balance between stability and adaptability. This design allows the lens 20 to work in harmony with the eye's natural mechanisms, offering a sophisticated solution for correcting refractive errors while prioritizing long-term eye health and functionality.

The innovative phakic intraocular lens design may represent a significant advancement in vision correction, potentially offering comprehensive treatment for multiple refractive conditions. This lens 20 not only can correct ametropia (refractive errors such as myopia and hyperopia) but can also effectively address astigmatism through its combination of toric design and stable positioning mechanism. The lens's haptic system, featuring concave edges and rounded corner lobes that anchor securely within the sulcus angle, can provide rotational (e.g., azimuthal) stability for effective toric correction.

Accordingly, various phakic refractive lens designs described herein provide the ability to maintain precise rotational alignment through its haptic mechanism while preserving axial movement with aqueous flow. This dual functionality enables stable toric correction for astigmatism while maintaining the benefits of a floating design, significantly reducing risks associated with fixed lens designs such as increased intraocular pressure, pigment dispersion, and cataract formation. Various lenses 20 described herein effectively combine the advantages of toric correction with the safety features of dynamic positioning, offering a more comprehensive solution for patients with complex refractive needs.

Likewise, posterior chamber floating phakic refractive lenses are provided that can address the challenges of maintaining stability and alignment within the dynamic environment of the eye. Such lenses may features a thin, rectangular-like shaped, buoyant structure that floats in the aqueous humor while preserving flexibility and softness. Its membrane-like design allows the lens to dynamically adapt to the eye's natural movements and the flow of aqueous humor, providing suitable Z-axis positioning and maintaining a gap between the phakic lens and the natural crystalline lens 14.

A small central hole 44 in the lens optic may facilitate the flow of aqueous humor through the lens, creating a centering force that stabilizes the lens within the pupillary space. Various such designs also may eliminate risks associated with rotational misalignment or decentration, as the lens prioritizes axial movement for enhanced safety and functionality. The lens's flexibility further aids in distributing mechanical stresses evenly, reducing the risk of pressure-induced damage to delicate ocular structures.

By integrating features such as dynamic axial adaptation, enhanced flexibility, and a stable floating mechanism, various such designs may represent a significant advancement in phakic lens technology, potentially offering a sophisticated solution for correcting refractive errors while providing long-term compatibility with the eye's natural physiology.

In various designs, any one or more of, for example, most or all of the surfaces and/or perimeter(s) and/or edge(s) 29, 30, 66 and/or corners 52, 54, 60 of the lens 20 and/or optical zone 22, and/or 60%, 70%, 80%, 90%, 95%, 97%, 98%, 99%, 100%, of surface area or length of any one or of these, or any range formed by any of the values, are smooth and/or continuous, e.g., without seams, steps, ridges, discontinuities, interruptions, corrugation, undulation, sharp points or features, or any one or more of these. Such smooth and/or continues perimeters, edges, and corners of the lens 20 and/or optical zone 22 reduce damage to the internal structure of the eye 10. In various designs, for example, most or all or 60%, 70%, 80%, 90%, 95%, 97%, 98%, 99% or any range formed by any of the values, of the corners 52, 54, 60 (e.g., as seen from the top or front view) have a radius of curvature of at least 0.1 mm, 0.2 mm, 0.3 mm, 0.4 mm, 0.5 mm, 0.6 mm, 0.7 mm, 0.8 mm, 0.9 mm, 1.0 mm, 1.1 mm, or any range formed by any of these values or possibly larger or smaller, for example, in the range from 0.3 mm to 0.7 mm, e.g., 0.5 mm for lenses such as having the sizes described herein. Other features may contribute to the smooth nature of the lens 10, e.g., of the surfaces and/or perimeter(s) and/or edges of the lens.

Although the non-rotationally symmetric or rotationally asymmetric surface configured to provide for astigmatic correction may be on the anterior (and not posterior) side or surface of the lens such as of the optical zone 22, in some implementations, the non-rotationally symmetric or rotationally asymmetric surface configured to provide for astigmatic correction is on the posterior (and not anterior) side or surface of the lens such as of the optical zone 22. In some implementations, however, both the anterior side or surface of the lens such as the optical zone 22 and the posterior side or surface of the lens such as the optical zone 22 comprises non-rotationally symmetric or rotationally asymmetric surfaces that provide for astigmatic correction.

Also, although various geometric features have been described as has having radii of curvature and thus may have circular or spherical shapes, in various designs, the curvature deviates from being purely circular or spherical in shape. In various such cases, however, the curvature may nevertheless be within a range of radii of curvatures such as, for example, set forth herein. For example, either or both the curved edge 30 or the indentation 34 need not have a circular shape (e.g., as seen from the top or front view), but may nevertheless be within a range of radii of curvatures such as, for example, set forth herein.

### EXAMPLES

The following is a numbered list of examples that are within the scope of this disclosure. The example designs and implementations that are listed should in no way be interpreted as limiting the scope of the possibilities. Various features of the example designs that are listed can be removed, added, or combined to form additional implementations, which are part of this disclosure.

### Part I

1. A phakic refractive lens comprising:
   a lens body having first and second longitudinally spaced ends and first and second laterally spaced edges, said body having anterior and posterior sides and a thickness therebetween, said body curved such that said posterior side is concave, said body comprising material that is buoyant in aqueous humor;
   an optical zone centrally located within said lens body, said optical zone being transparent and curved so as to refract light incident on the anterior side thereof thereby providing refractive optical power, said optical zone having a non-rotationally symmetric anterior surface with different first and second curvatures along different first and second radial directions such that different optical power is provided along different directions;
   a hole extending from an anterior side to a posterior side of the phakic refractive lens; and
   at least one haptic extending to an end of said lens body, the haptic including first and second rounded lobes on each of said first and second longitudinally spaced ends,
   wherein said lens body has a lateral width that is narrower closer to said optical zone than at the widest part of the lens at said rounded lobes at said first end, said first and second laterally spaced edges having reduced distance therebetween at the center of said phakic refractive lens and increased distance therebetween at the widest separation between the first and second laterally spaced edges at said first and second rounded lobes at said first end.
2. The phakic refractive lens of Example 1, wherein said non-rotationally symmetric anterior surface comprises a toric surface.
3. The phakic refractive lens of Example 1 or 2, wherein said first and second laterally spaced edges are curved.
4. The phakic refractive lens of any of the examples above, wherein said first and second laterally spaced edges are concave.
5. The phakic refractive lens of any of the examples above, wherein said first and second edges have a radius of curvature of from 8 to 12 millimeters (mm).
6. The phakic refractive lens of any of the examples above, wherein phakic refractive lens is a monolithic structure.
7. The phakic refractive lens of any of the examples above, wherein said material comprising said lens body comprises a homogonous composition throughout.
8. The phakic refractive lens of any of the examples above, wherein said material comprising said lens body has a specific gravity of from 0.9 to 1.2 g/cm³.
9. The phakic refractive lens of any of the examples above, wherein said material comprises silicone, silicone polymers, poly(acrylates), poly(methacrylates), hydrogels, proteins, collagens, copolymers, or mixtures thereof.
10. The phakic refractive lens of any of the examples above, wherein said material has a hardness of from 20 to 60 Shore A.
11. The phakic refractive lens of any of the examples above, further comprising an indentation at an end of said lens body between said first and second rounded lobes.
12. The phakic refractive lens of Example 11, wherein said indentation is curved.
13. The phakic refractive lens of Example 12, wherein said indentation has a radius of curvature of from 2 to 3 mm.
14. The phakic refractive lens of any of the examples above, wherein said hole comprises at least one hole in at least one of said first and second rounded lobes.
15. The phakic refractive lens of Example 14, wherein said at least one hole in at least one of said rounded lobes is from 0.5 to 1 millimeter wide.
16. The phakic refractive lens of Example 14, wherein said at least one hole is from 1.0 mm to 2 mm from the end of said rounded lobe.
17. The phakic refractive lens of any of the examples above, wherein said optical zone is circular.
18. The phakic refractive lens of any of the examples above, wherein said hole is in said optical zone.
19. The phakic refractive lens of Example 17, wherein said hole in said optical zone is at the center of said optical zone.
20. The phakic refractive lens of any of Examples 17-18, wherein the lens body has a thickness at said hole in said optical zone of from 0.025 mm to 0.075 mm.
21. The phakic refractive lens of any of the examples above, wherein said phakic refractive lens is thicker at at least a portion of said optical zone than at said end of said lens body.
22. The phakic refractive lens of any of the examples above, wherein said lens body has a thickness at the first and second rounded lobes of 0.075 to 0.125 millimeters.
23. The phakic refractive lens of any of the examples above, wherein said optical zone protrudes in the anterior direction with respect to the anterior side of said haptic.
24. The phakic refractive lens of any of the examples above, further comprising a transition zone extending from said optical zone to said haptic.
25. The phakic refractive lens of Example 24, wherein said transition zone has a curved edge adjacent said optical zone.
26. The phakic refractive lens of Example 24 or 25, wherein said transition has a curved edge adjacent said haptic.
27. The phakic refractive lens of any of the examples above, wherein said first and second longitudinally spaced ends of said lens body terminate in rounded thicknesses.
28. The phakic refractive lens of Example 27, wherein said rounded thicknesses has a radius of curvature of 0.025 mm to 0.045 mm.
29. The phakic refractive lens of any of the examples above, wherein said posterior side of said lens body has a radius of curvature of from 7.5 to 13.5 mm.
30. The phakic refractive lens of any of the examples above, wherein said lens body has a length along a longitudinal axis of the phakic refractive lens between the farthest extensions of said first and second longitudinally spaced ends of said lens body of from 12.2 to 13.2 mm.
31. The phakic refractive lens of any of the examples above, wherein the farthest distance of a first haptic on said first end of said lens body to a second haptic on said second end of said lens body is from 12.8 to 13.8 mm.
32. The phakic refractive lens of any of the examples above, wherein said lens body has an aspect ratio of length along a longitudinal axis of said phakic refractive lens between the farthest extensions of first and second longitudinally spaced ends of said lens body to width along a lateral axis of said phakic refractive lens between the farthest extensions of said laterally spaced edges of said lens body of from 1.4 to 2.0.
33. The phakic refractive lens of any of the examples above, wherein said hole is disposed in said optical zone and has a width of from 0.2 mm to 0.6 mm.
34. The phakic refractive lens of any of the examples above, wherein said rounded lobe has a distal end thereof that has a radius of curvature of from 5 mm to 8 mm.
35. The phakic refractive lens of any of the examples above, wherein said rounded lobe has a central region with a larger radius of curvature and corners with a smaller radius of curvature.
36. The phakic refractive lens of any of the examples above, wherein lens body comprises a membrane.
37. The phakic refractive lens of any of the examples above, wherein said lens body has a lateral width that is narrower closer to said optical zone than at the widest lateral separation between said rounded lobes at said second end, said first and second laterally spaced edges having reduced distance therebetween at the center of said phakic refractive lens and increased distance therebetween at the widest separation between said first and second rounded lobes at said second end.

### Part II

1. A phakic refractive lens having first and second longitudinally spaced ends and first and second laterally spaced edges, said phakic refractive lens having first and second sides and a thickness therebetween, said phakic refractive lens comprising:
   an optical zone centrally located between said first and second longitudinally spaced ends, said optical zone being transparent and curved so as to refract light incident on the first side thereof thereby providing refractive optical power, said optical zone having a non-rotationally symmetric first surface with different first and second curvatures along different first and second radial directions such that different optical power is provided along different directions;
   a hole configured to allow aqueous humor to flow therethrough between said first and second sides of the phakic refractive lens; and
   a support extending to said first end and/or said second end of said phakic refractive lens, respectively, the support including first and second rounded lobes at first and second corners at one or both of said first and second haptics,
   wherein said phakic refractive lens comprises material that is buoyant in aqueous humor.
2. The phakic refractive lens of Example 1, wherein said phakic refractive lens is curved such that said second side is concave, said second side configured to face posteriorly when implanted.
3. The phakic refractive lens of Example 1 or 2, wherein said phakic refractive lens has a lateral width that is narrower closer to said optical zone than at the widest lateral separation between said rounded lobes at said second end, said first and second laterally spaced edges having reduced distance therebetween at the center of said phakic refractive lens and increased distance therebetween at said widest separation between said first and second rounded lobes.
4. The phakic refractive lens of Example 1 or 2, wherein said phakic refractive lens has a lateral width that is narrower closer to said optical zone than at the widest lateral separation between said outer perimeter of the rounded lobes at said second end, said first and second laterally spaced edges having reduced distance therebetween at the center of said phakic refractive lens and increased distance therebetween at the widest separation between the outer perimeter of said first and second rounded lobes at said second end.
5. The phakic refractive lens of Example 4, wherein said phakic refractive lens has a lateral width that is narrower closer to said optical zone than at the widest lateral separation between said outer perimeter of the rounded lobes at said first end, said first and second laterally spaced edges having reduced distance therebetween at the center of said phakic refractive lens and increased distance therebetween at the widest separation between the outer perimeter of said first and second rounded lobes at said first end.
6. The phakic refractive lens of any of the examples above, wherein said non-rotationally symmetric first surface comprises a toric surface.
7. The phakic refractive lens of any of the examples above, wherein said first and second laterally spaced edges are curved.
8. The phakic refractive lens of any of the examples above, wherein said first and second laterally spaced edges are concave.
9. The phakic refractive lens of any of the examples above, wherein said first and second edges have a radius of curvature of from 7 to 13 millimeters.
10. The phakic refractive lens of any of the examples above, wherein phakic refractive lens is a monolithic structure.

### Part III

1. A phakic refractive lens having first and second longitudinally spaced ends and first and second laterally spaced edges, said phakic refractive lens having first and second sides and a thickness therebetween, said phakic refractive lens comprising:
   an optical zone centrally located between said first and second longitudinally spaced ends, said optical zone being transparent and curved so as to refract light incident on the first side thereof thereby providing refractive optical power;
   a hole configured to allow aqueous humor to flow therethrough between first and second sides of the phakic refractive lens; and
   a support extending to said first end and/or said second end of said phakic refractive lens, respectively,
   wherein said phakic refractive lens comprises material that is buoyant in aqueous humor, and
   wherein said phakic refractive lens has a lateral width that is narrower closer to said optical zone and wider farther from said optical zone and closer to at least one of said first and second longitudinally spaced ends.
2. The phakic refractive lens of Example 1, wherein said optical zone has a non-rotationally symmetric first surface with different first and second curvatures along different first and second radial directions such that different optical power is provided along different directions.
3. The phakic refractive lens Example 2, wherein said non-rotationally symmetric surface comprises a toric surface.
4. The phakic refractive lens of any of the examples above, wherein the support includes first and second rounded lobes at said first and second longitudinally spaced ends of the phakic refractive lens.
5. The phakic refractive lens of Example 4, wherein said lens has a lateral width that is narrower closer to said optical zone than at the widest lateral separation between said rounded lobes at said first end, said first and second laterally spaced edges having reduced distance therebetween at the center of said phakic refractive lens and increased distance therebetween at the widest separation between said first and second rounded lobes at said first end.
6. The phakic refractive lens of Example 5, wherein said lens has a lateral width that is narrower closer to said optical zone than at the widest lateral separation between said rounded lobes at said second end, said first and second laterally spaced edges having reduced distance therebetween at the center of said phakic refractive lens and increased distance therebetween at the widest separation between said first and second rounded lobes at said second end.
7. The phakic refractive lens of any of Examples 4-6, further comprising an indentation at an end of said lens between said first and second rounded lobes.
8. The phakic refractive lens of Example 7, wherein said indentation is curved.
9. The phakic refractive lens of Example 8, wherein said indentation has a radius of curvature of from 2 to 3 mm.
10. The phakic refractive lens of any of Examples 4-9, wherein said hole comprises at least one hole in at least one of said first and second rounded lobes.
11. The phakic refractive lens of Example 10, wherein said at least one hole in at least one of said rounded lobes is from 0.5 to 1 millimeter wide.
12. The phakic refractive lens of Example 10, wherein said at least one hole is from 1.0 mm to 2 mm from the end of said rounded lobe.
13. The phakic refractive lens of any of Examples 4-12, wherein said phakic refractive lens has a thickness at the first and second rounded lobes of 0.075 to 0.125 millimeters.
14. The phakic refractive lens of any of Examples 4-13, wherein said rounded lobe has a distal end thereof that has a radius of curvature of from 5 mm to 8 mm.
15. The phakic refractive lens of any of Examples 4-14, wherein said rounded lobe has a central region with a larger radius of curvature and corners with a smaller radius of curvature.
16. The phakic refractive lens of any of the examples above, wherein said first and second laterally spaced edges are curved.
17. The phakic refractive lens of any of the examples above, wherein said first and second laterally spaced edges are concave.
18. The phakic refractive lens of any of the examples above, wherein said first and second edges have a radius of curvature of from 8 to 12 millimeters (mm).
19. The phakic refractive lens of any of the examples above, wherein phakic refractive lens is a monolithic structure.
20. The phakic refractive lens of any of the examples above, wherein said material comprising said lens comprises a homogonous composition throughout.
21. The phakic refractive lens of any of the examples above, wherein said material comprising said lens has a specific gravity of from 0.9 to 1.2 g/cm³.
22. The phakic refractive lens of any of the examples above, wherein said material comprises silicone, silicone polymers, poly(acrylates), poly(methacrylates), hydrogels, proteins, collagens, copolymers, or mixtures thereof.
23. The phakic refractive lens of any of the examples above, wherein said material has a hardness of from 20 to 60 Shore A.
24. The phakic refractive lens of any of the examples above, wherein said optical zone is circular.
25. The phakic refractive lens of any of the examples above, wherein said hole is in said optical zone.
26. The phakic refractive lens of Example 25, wherein said hole in said optical zone is at the center of said optical zone.
27. The phakic refractive lens of any of Examples 25-26, wherein the lens has a thickness at said hole in said optical zone of from 0.025 mm to 0.075 mm.
28. The phakic refractive lens of any of the examples above, wherein said hole is disposed in said optical zone and has a width of from 0.2 mm to 0.6 mm.
29. The phakic refractive lens of any of the examples above, wherein said phakic refractive lens is thicker at at least a portion of said optical zone than at said end of said lens.
30. The phakic refractive lens of any of the examples above, wherein said optical zone protrudes in the anterior direction with respect to the anterior side of said support.
31. The phakic refractive lens of any of the examples above, further comprising a transition zone extending from said optical zone to said support.
32. The phakic refractive lens of Example 31, wherein said transition zone has a curved edge adjacent said optical zone.
33. The phakic refractive lens of Example 31 or 32, wherein said transition has a curved edge adjacent said support.
34. The phakic refractive lens of any of the examples above, wherein said posterior side of said lens has a radius of curvature of from 7.5 to 13.5 mm.
35. The phakic refractive lens of any of the examples above, wherein said lens has a length along a longitudinal axis of the phakic refractive lens between the farthest extensions of said first and second longitudinally spaced ends of said lens of from 12.2 to 13.2 mm.
36. The phakic refractive lens of any of the examples above, wherein said lens has an aspect ratio of length along a longitudinal axis of said phakic refractive lens between the farthest extensions of first and second longitudinally spaced ends of said lens to width along a lateral axis of said phakic refractive lens between the farthest extensions of said laterally spaced edges of said lens of from 1.4 to 2.0.
37. The phakic refractive lens of any of the examples above, wherein first and second longitudinally spaced ends of said lens terminate in rounded thicknesses.
38. The phakic refractive lens of Example 37, wherein said rounded thicknesses has a radius of curvature of 0.025 mm to 0.045 mm.
39. The phakic refractive lens of any of the examples above, wherein the farthest distance of the support on said first end of said lens to a support on said second end of said membrane is from 12.8 to 13.8 mm.

Although the description above contains many details and specifics, these should not be construed as limiting the scope of the invention but as merely providing illustrations of some of the presently preferred embodiments of this invention. Other implementations, enhancements and variations can be made based on what is described and illustrated in this patent document. The features of the embodiments described herein may be combined in all possible combinations of methods, apparatus, modules, systems, and computer program products. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination. Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described above should not be understood as requiring such separation in all embodiments.

Therefore, it will be appreciated that the scope of the present invention fully encompasses other embodiments which may become obvious to those skilled in the art. In the claims, reference to an element in the singular is not intended to mean “one and only one” unless explicitly so stated, but rather “one or more.” All structural and functional equivalents to the elements of the above-described preferred embodiment that are known to those of ordinary skill in the art are expressly incorporated herein by reference and are intended to be encompassed by the present claims. Moreover, it is not necessary for a device to address each and every problem sought to be solved by the present invention, for it to be encompassed by the present claims. Furthermore, no element or component in the present disclosure is intended to be dedicated to the public regardless of whether the element or component is explicitly recited in the claims. No claim element herein is to be construed under the provisions of 35 U.S.C. 112, sixth paragraph, unless the element is expressly recited using the phrase “means for.”

What is claimed is:

1. A phakic refractive lens comprising:
a lens body having first and second longitudinally spaced ends and first and second laterally spaced edges, said body having anterior and posterior sides and a thickness therebetween, said body curved such that said posterior side is concave, said body comprising material that is buoyant in aqueous humor;
an optical zone centrally located within said lens body, said optical zone being transparent and curved so as to refract light incident on the anterior side thereof thereby providing refractive optical power, said optical zone having a non-rotationally symmetric anterior surface with different first and second curvatures along different first and second radial directions such that different optical power is provided along different directions;
a hole extending from an anterior side to a posterior side of the phakic refractive lens; and
at least one haptic extending to an end of said lens body, the haptic including first and second rounded lobes on each of said first and second longitudinally spaced ends,
wherein said lens body has a lateral width that is narrower closer to said optical zone than at the widest lateral separation between said rounded lobes at said first end, said first and second laterally spaced edges having reduced distance therebetween at the center of said phakic refractive lens and increased distance therebetween at the widest separation between said first and second rounded lobes at said first end.

2. The phakic refractive lens of claim 1, wherein said non-rotationally symmetric anterior surface comprises a toric surface.

3. The phakic refractive lens of claim 1, wherein said first and second laterally spaced edges are curved.

4. The phakic refractive lens of claim 1, wherein said first and second laterally spaced edges are concave.

5. The phakic refractive lens of claim 1, wherein said first and second edges have a radius of curvature of from 8 to 12 millimeters (mm).

6. The phakic refractive lens of claim 1, wherein phakic refractive lens is a monolithic structure.

7. The phakic refractive lens of claim 1, wherein said material comprising said lens body has a specific gravity of from 0.9 to 1.2 g/cm³.

8. The phakic refractive lens of claim 1, wherein said material has a hardness of from 20 to 60 Shore A.

9. The phakic refractive lens of claim 1, further comprising an indentation at an end of said lens body between said first and second rounded lobes.

10. The phakic refractive lens of claim 9, wherein said indentation is curved.

11. The phakic refractive lens of claim 1, wherein said hole comprises at least one hole in at least one of said first and second rounded lobes.

12. The phakic refractive lens of claim 1, wherein said hole is in said optical zone.

13. The phakic refractive lens of claim 1, further comprising a transition zone extending from said optical zone to said haptic.

14. The phakic refractive lens of claim 1, wherein said lens body has a length along a longitudinal axis of the phakic refractive lens between the farthest extensions of said first and second longitudinally spaced ends of said lens body of from 12.2 to 13.2 mm.

15. The phakic refractive lens of claim 1, wherein the farthest distance of a first haptic on said first end of said lens body to a second haptic on said second end of said lens body is from 12.8 to 13.8 mm.
